(19) United States
(12) Patent Application Publication
(10) Pub. No.: US 2003/0131843 A1
(43) Pub. Date: Jul. 17, 2003

(54) Open-celled substrates for drug delivery

(76) Inventor: Amy T. Lu, Los Altos, CA (US)

Correspondence Address: Richard E. Eckman, Morrison & Foerster LLP, 755 Page Mill Road, Palo Alto, CA, 94304-1018, US

(21) Appl. No.: 10/302,614

(22) Filed: Nov. 21, 2002

Related U.S. Application Data
(60) Provisional application No. 60/332,165, filed on Nov. 21, 2001.

(51) Int. Cl. A61M 16/00

(52) U.S. Cl. 128/200.24; 128/202.21; 128/203.26

(57) ABSTRACT

The present invention relates to the delivery of drugs through an inhalation route. Specifically, it relates to the formation of drug thermal vapors from a heated open-celled substrate for use in inhalation therapy. In a method aspect of the present invention, a method of delivering a drug to a mammal through an inhalation route is provided which comprises heating a composition to form a thermal vapor, which is inhaled by the mammal, wherein the composition comprises a drug, and wherein the composition is coated onto a substrate, and wherein the substrate has a high surface to volume ratio, high porosity, and a three-dimensional network of interconnected cells. In a device aspect of the present invention, a device for delivering a drug to a mammal through an inhalation route is provided, wherein the device comprises: a power source; a substrate, wherein the substrate has a high surface to volume ratio, high porosity, and a three-dimensional network of interconnected cells; and, an element permitting the mammal to inhale the thermal vapor. In a kit aspect of the present invention, a kit for delivering a drug to a mammal through an inhalation route is provided which comprises: a) a composition comprising a drug; and b) a device that forms a drug thermal vapor from the composition for inhalation by the mammal, wherein the device comprises a substrate, and wherein the substrate has a high surface to volume ratio, high porosity, and a three-dimensional network of interconnected cells.

### CROSS-REFERENCE TO RELATED APPLICATIONS

This application claims priority to U.S. provisional application Serial No. 60/332,165 entitled “Open-celled substrates for drug delivery,” filed Nov. 21, 2001, Amy T. Lu, the entire disclosure of which is hereby incorporated by reference.

### FIELD OF THE INVENTION

The present invention relates to the delivery of drugs through an inhalation route. Specifically, it relates to the formation of drug thermal vapors from a heated open-celled substrate for use in inhalation therapy.

### BACKGROUND OF THE INVENTION

There are several methods discussed in the literature for delivering a drug through an inhalation route. Breath actuated inhalers, for instance, typically contain a pressurized propellant that provides a metered dose of drug upon a patient's inspiration. Dry powder formulations are delivered using a compressed charge of air to disperse drug powder into an aerosol cloud. For certain drugs, volatilization by heating has been proposed as an administration method.

WO 94/09842 (“Rosen”) discusses coating a layer of pharmaceutically active drug on the surface of an electrically conductive metal. Rosen suggests that passing a current through the metal will generate heat, thereby converting drug to an inhalable gaseous phase. U.S. Pat. No. 4,922,901 (“Brooks”) proposes providing a dose of drug in aerosol form using a drug delivery article having an electrical resistance heating element and an electrical power source. Brooks states that the heating element preferably carries one or more aerosol forming substances.

Neither Rosen nor Brooks discuss an open-celled substrate from which a drug can be volatilized. The provision of such a substrate is an object of the present invention.

### SUMMARY OF THE INVENTION

The present invention relates to the delivery of drugs through an inhalation route. Specifically, it relates to the formation of drug thermal vapors from a heated, open-celled substrate for use in inhalation therapy.

In a method aspect of the present invention, a method of delivering a drug to a mammal through an inhalation route is provided which comprises heating a composition to form a thermal vapor, which is inhaled by the mammal, wherein the composition comprises a drug, and wherein the composition is coated onto a substrate, and wherein the substrate has a high surface to volume ratio, high porosity, and a three-dimensional network of interconnected cells.

Typically, the substrate has about 5, 10, 20, 30, 40, 45, 50, 60, 70, 80, 90 or 100 pores per linear inch.

Typically, the relative density of the substrate is 3% to 30% or 3% to 12%.

Typically, the substrate has a surface to volume ratio greater than 300/ft, 400/ft, 500/ft, 600/ft, 700/ft, 800/ft, 900/ft, 1000/ft, 1100/ft, 1200/ft, 1300/ft, 1400/ft or 1500/ft.

Typically, the nominal resistance to air flow for a substrate is less than 0.5, 1.0, 1.5, 2.0, 2.5, 3.0, 3.5, 4.0 or 4.5.

Typically, the substrate is heated by passing current through it.

In a device aspect of the present invention, a device for delivering a drug to a mammal through an inhalation route is provided, wherein the device comprises: a power source; a substrate, wherein the substrate has a high surface to volume ratio, high porosity, and a three-dimensional network of interconnected cells; and, an element permitting the mammal to inhale the thermal vapor.

Typically, the substrate has about 5, 10, 20, 30, 40, 45, 50, 60, 70, 80, 90 or 100 pores per linear inch.

Typically, the relative density of the substrate is 3% to 30% or 3% to 12%.

Typically, the substrate has a surface to volume ratio greater than 300/ft, 400/ft, 500/ft, 600/ft, 700/ft, 800/ft, 900/ft, 1000/ft, 1100/ft, 1200/ft, 1300/ft, 1400/ft or 1500/ft.

Typically, the nominal resistance to air flow for a substrate is less than 0.5, 1.0, 1.5, 2.0, 2.5, 3.0, 3.5, 4.0 or 4.5.

Typically, the substrate is a resistive heating element.

In a kit aspect of the present invention, a kit for delivering a drug to a mammal through an inhalation route is provided which comprises: a) a composition comprising a drug; and b) a device that forms a drug thermal vapor from the composition for inhalation by the mammal, wherein the device comprises a substrate, and wherein the substrate has a high surface to volume ratio, high porosity, and a three-dimensional network of interconnected cells.

Typically, the substrate has about 5, 10, 20, 30, 40, 45, 50, 60, 70, 80, 90 or 100 pores per linear inch.

Typically, the relative density of the substrate is 3% to 30% or 3% to 12%.

Typically, the substrate has a surface to volume ratio greater than 300/ft, 400/ft, 500/ft, 600/ft, 700/ft, 800/ft, 900/ft, 1000/ft, 1100/ft, 1200/ft, 1300/ft, 1400/ft or 1500/ft.

Typically, the nominal resistance to air flow for a substrate is less than 0.5, 1.0, 1.5, 2.0, 2.5, 3.0, 3.5, 4.0 or 4.5.

Typically, the drug composition is coated on the substrate.

Typically, the substrate is a resistive heating element.

### BRIEF DESCRIPTION OF THE FIGURE

FIG. 1 shows a device comprising an open-celled substrate used to deliver drug thermal vapors to a mammal through an inhalation route.

### DETAILED DESCRIPTION OF THE INVENTION

“Aerosol” refers to a suspension of solid or liquid particles in a gas.

“Condensation aerosol” refers to an aerosol formed by vaporization of a substance followed by condensation of the substance into an aerosol.

“Nominal resistance to air flow” refers to the pressure drop in units of inches H₂O across a 10″ diameter×1″ thick substrate with an air velocity of 600 feet per minute.

“Relative density” refers to the percent solid, or the volume of solid material relative to void space in the substrate.

“Vapor” refers to a gas, and “vapor phase” refers to a gas phase. The term “thermal vapor” refers to a vapor phase, aerosol, or mixture of aerosol-vapor phases, formed preferably by heating.

The open-celled substrates of the present invention have a high surface to volume ratio, high porosity, and a three-dimensional network of interconnected cells. The substrates are formed from carbonaceous materials, mixtures of carbonaceous materials, non-carbonaceous materials, mixtures of non-carbonaceous materials (e.g., metal plated materials or alloys) or a mixture of carbonaceous and non-carbonaceous materials. Examples of materials used to form the substrates include, without limitation, vitreous carbon, silicon carbide, aluminum, copper, gold, silver, nickel chromium alloy and gold deposited on vitreous carbon.

Open-celled substrates are either obtained commercially or manufactured. For instance, such substrates are available from Energy Research and Generation, Inc. (Oakland, Calif.). Manufacturing routes are generally described in Barnhart, J., Manufacturing Routes for Metallic Foams, JOM, 52(12) (2000), pp. 22-27. Where the substrate comprises, for example, gold deposited on reticulated vitreous carbon, the gold is typically deposited using standard methods in the art, such as chemical vapor deposition or electrochemical plating.

The substrates of the present invention are of a variety of shapes and designs. Examples of such shapes include, without limitation, cylinders and boxes. The substrate is either bonded to another substrate or not. For instance, the open-celled substrate, in certain cases, is bonded or adhered to a second substrate (e.g., a copper cylinder).

Any suitable method is used to form thermal vapors for drug delivery using the substrates of the present invention. A preferred method, however, involves the following steps: coating the substrate with a composition comprising a drug; heating the substrate to produce a drug containing vapor; and, allowing the vapor to cool such that it condenses to provide a condensation aerosol.

The composition is generally heated in one of two forms: as pure drug; or as a mixture of pure drug and a pharmaceutically acceptable excipient. Pharmaceutically acceptable excipients are either volatile or nonvolatile. Volatile excipients, when heated, are concurrently volatilized and inhaled with the drug. Classes of such excipients are known in the art and include, without limitation, gaseous, supercritical fluid, liquid and solid solvents. The following is a list of exemplary carriers within the classes: water; terpenes, such as menthol; alcohols, such as ethanol, propylene glycol, glycerol and other similar alcohols; dimethylformamide; dimethylacetamide; wax; supercritical carbon dioxide; dry ice; and mixtures thereof.

Nonlimiting examples of drugs that are delivered from a heated open-celled substrate for use in inhalation therapy include the following: acetaminophen, alfenatil, alprazolam, amantadine, amitriptyline, amobarbital, amoxipine, aspirin, astemizole, atenolol, azatidine, baclofen, benztropine mesylate, beta estradiol, betahistine, biperiden, bromazepam, bromocryptine, brompheniramine, buprenorphine, bupropion, buspirone, butalbital, butorphanol, caffeine, carbamazepine, carbidopa, carisoprodol, celecoxib, cetirizine, chloral hydrate, chlordiazepoxide, chlorpheniramine, chlorpromazine, chlorzoxazone, cinnarizine, citalopram, clemastine, clofazimine, clomipramine, clonazepam, clonidine, clorazepate, clozapine, codeine, cyclobenzaprine, cyproheptadine, desipramine, dextroamphetamine, dezocine, diazepam, diclofenac, diclofenac ethyl ester, diflunisal, dihydroergotamine, dimenhydrinate, diphenhydramine, disulfiram, dolasetron, doxepin, doxylamine, dronabinol, droperidol, entacapone, ergotamine, estazolam, estradiol 17-enanthate, ethosuximide, etodolac, felbamate, fenoprofen, fentanyl, flunitrazepam, fluoxetine, fluphenazine, flurazepam, fluribiprofen, fluvoxamine, fosphenytoin, gabapentin, granisetron, haloperidol, hydrocodone, hydromorphone, hydroxyzine, hyoscyamine, ibuprofen, imipramine, indomethacin, isocarboxazid, ketoprofen, ketoprofen ethyl ester, ketorolac, ketorolac ethyl ester, ketorolac methyl ester, lamotrigine, levetiracetam, levodopa, levorphenol, lithium, lorazepam, loxapine, maprotiline, meclizine, meclofenamate, meloxicam, meperidine, mephobarbital, meprobamate, mesoridazine, metaxalone, methadone, methocarbamol, methsuximide, methylphenidate, methylprednisolone, methysergide, metoclopramide, midazolam, mirtazapine, modafinil, molindone, morphine, nabumetone, nalbuphine, nalmefene, naloxone, naltrexone, naproxen, naratriptan, nefazodone, nicotine, nortriptyline, olanzapine, ondansetron, orphenadrine, oxaprozin, oxazepam, oxcarbazepine, oxybutynin, oxycodone, oxymorphone, paroxetine, pemoline, pentazocine, pentobarbital, pergolide, perphenazine, phenelzine, phenobarbital, phentermine, phenytoin, pimozide, pindolol, piroxicam, pramipexole, pregnanalone, primidone, prochlorperazine, promethazine, propoxyphene, protriptyline, pyrilamine, quetiapine, quinine, rauwolfia, remifentanil, risperidone, rizatriptan, rofecoxib, ropinirole, salsalate, scopolamine, secobarbital, selegiline, sertraline, sibutramine, sildenafil, sufentanil, sulindac, sumatriptan, temazepam, testosterone, thioridazine, thiothixene, tiagabine, tizanidine, tolcapone, tolfenamic acid, tolmetin, topiramate, tramadol, tranylcypromine, trazodone, triazolam, trichlormethiazide, trifluoperazine, trihexyphenidyl, trimethobenzamide, trimipramine, valproic acid, venlafaxine, zaleplon, zolmitriptan, zolpidem, zonisamide, and zopiclone.

The composition is coated onto the substrate using a number of different methods. Such methods include, without limitation, adding a solution of the drug in a volatile organic solvent to the substrate and allowing the solvent to evaporate; dipping the substrate into a solution of drug in a volatile organic solvent, removing it and allowing the solvent to evaporate; depositing the compound through chemical vapor deposition.

Typically, the substrate is heated by placing electrodes at either end and passing an electric current through it (i.e., resistive heating). Alternatively, the substrate can be bonded to a second substrate that is heated. Heating then occurs through thermal conductivity pathways. Examples of methods by which the second substrate can be heated include the following: passage of current through an electrical resistance element; absorption of electromagnetic radiation, such as microwave or laser light; and, exothermic chemical reactions, such as exothermic salvation, hydration of pyrophoric materials and oxidation of combustible materials.

Drug containing thermal vapors formed from the substrate are delivered to a mammal using an inhalation device. Where the thermal vapor is a condensation aerosol, the device has at least three elements: an open-celled substrate that heats a drug containing composition to form a vapor; an element allowing the vapor to cool, thereby providing a condensation aerosol; and, an element permitting the mammal to inhale the aerosol. Various suitable heating methods are described above. The element that allows cooling is, in it simplest form, an inert passageway linking the heating means to the inhalation means. The element permitting inhalation is an aerosol exit portal that forms a connection between the cooling element and the mammal's respiratory system.

An air flow typically carries the thermal vapor to the mammal's respiratory system. In certain devices, the air flow travels around the open-celled substrate from which the drug containing thermal vapor is being formed. The air flow travels through the substrate in others.

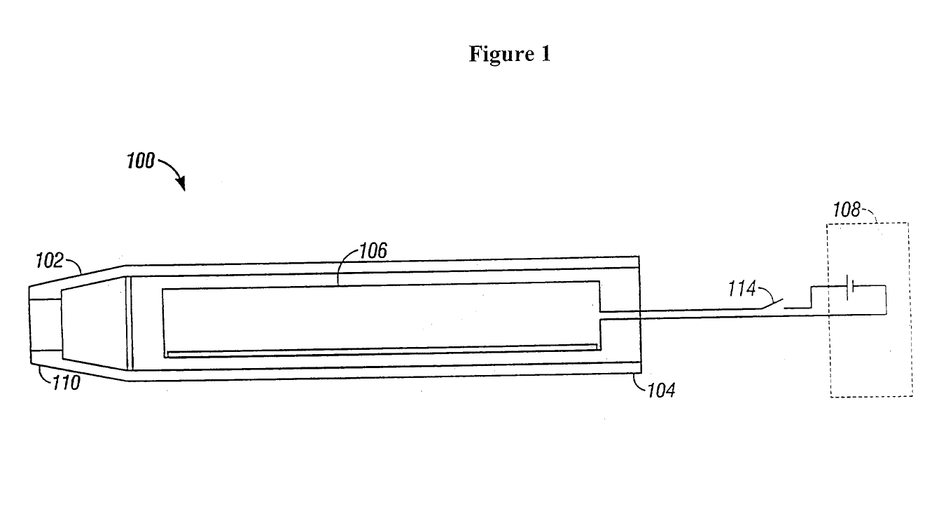

One device used to deliver drug containing thermal vapors is described in reference to FIG. 1. Delivery device 100 has a proximal end 102 and a distal end 104, an open-celled substrate 106, a power source 108, and a mouthpiece 110. A drug composition is deposited on substrate 106. Upon activation of a user activated switch 114, power source 108 initiates heating of substrate 106 through passage of current through it. The drug composition volatilizes due to the heating of substrate 106 and condenses to form a condensation aerosol prior to reaching the mouthpiece 110 at the proximal end of the device 102. Air flow traveling from the device distal end 104 to the mouthpiece 110 carries the condensation aerosol to the mouthpiece 110, where it is inhaled by the mammal.

A typical dosage of a thermal vapor is either administered as a single inhalation or as a series of inhalations taken within an hour or less (dosage equals sum of inhaled amounts). Where the drug is administered as a series of inhalations, a different amount may be delivered in each inhalation. The dosage amount of the drug in thermal vapor form is generally no greater than twice the standard dose of the drug given orally.

One can determine the appropriate dose of drug containing thermal vapors to treat a particular condition using methods such as animal experiments and a dose-finding (Phase I/II) clinical trial. One animal experiment involves measuring plasma concentrations of an animal after its exposure to the thermal vapor. Mammals such as dogs or primates are typically used in such studies, since their respiratory systems are similar to that of a human. Initial dose levels for testing in humans is generally less than or equal to the dose in the mammal model that resulted in plasma drug levels associated with a therapeutic effect in humans.. Dose escalation in humans is then performed, until either an optimal therapeutic response is obtained or a dose-limiting toxicity is encountered.

The following example is meant to illustrate, rather than limit, the present invention.

### EXAMPLE 1

### Volatilization of Diazepam on Reticulated Vitreous Carbon

A sample of reticulated vitreous carbon with a nominal pore size grade of 100 pores per linear inch was obtained from Energy Research and Generation, Inc. (Oakland, Calif.). The sample was cut into a stick with dimensions of about 0.64 cm×0.64 cm×3.0 cm. Solder was melted into two copper caps with dimensions (diameter×height) of 11.5 cm×10 cm to which a piece of copper wire had been soldered. The caps were placed on the ends of the reticulated vitreous carbon stick, and the solder was allowed to harden. Acetone was used to rinse the copper capped stick, which was then dried in a vacuum oven for about 0.5 h at 50° C. Diazepam (2.1 mg) in 360 μL dichloromethane was coated onto the exposed portions of the stick. The coated reticulated vitreous carbon was heated at 50° C. in vacuo to remove the dichloromethane. The stick was placed in a glass sleeve, with the attached copper wires protruding from either end, which was stoppered. The wires were connected to a 9 V battery. Aerosol generation began at about 9 s after connection to the battery. The battery connection was removed after a total of 15 s. Acetonitrile (2 mL) was used to rinse the inside of the glass sleeve after the stick had been removed. HPLC analysis with detection by light absorption at 225 nm showed that the diazepam (2.1 mg) volatilized in greater than 99.9% purity.

1. A method of delivering a drug to a mammal through an inhalation route comprising heating a composition to form a thermal vapor, which is inhaled by the mammal, wherein the composition comprises a drug, and wherein the composition is coated onto a substrate, and wherein the substrate has a high surface to volume ratio, high porosity, and a three-dimensional network of interconnected cells.

2. The method according to claim 1, wherein the substrate comprises pores, and wherein the number of pores per linear inch is between about 5 and about 100.

3. The method according to claim 2, wherein the relative density of the substrate is 3% to 30%

4. The method according to claim 3, wherein the substrate has a surface to volume ratio greater than 300/ft.

5. The method according to claim 4, wherein the nominal resistance to air flow for the substrate is less than 4.5.

6. The method according to claim 5, wherein the substrate is heated by passing current through it.

7. A device for delivering a drug to a mammal through an inhalation route, wherein the device comprises:
a) a power source;
b) a substrate connected to the power source, wherein the substrate has a high surface to volume ratio, high porosity, and a three-dimensional network of interconnected cells; and
c) an element permitting the mammal to inhale the thermal vapor.

8. The device according to claim 7, wherein the substrate comprises pores, and wherein the number of pores per linear inch is between about 5 and about 100.

9. The device according to claim 8, wherein the relative density of the substrate is 3% to 30%.

10. The device according to claim 9, wherein the substrate is a resistive heating element.

11. A kit for delivering a drug to a mammal through an inhalation route, wherein the kit comprises:
a) a composition comprising a drug; and
b) a device that forms a drug thermal vapor from the composition for inhalation by the mammal, wherein the device comprises a substrate, and wherein the substrate has a high surface to volume ratio, high porosity, and a three-dimensional network of interconnected cells.

12. The kit according to claim 11, wherein the substrate comprises pores, and wherein the number of pores per linear inch is between about 5 and about 100.

13. The kit according to claim 12, wherein the relative density of the substrate is 3% to 30%.

14. The kit according to claim 13, wherein the substrate is a resistive heating element.
